(19) United States
(12) Patent Application Publication
(10) Pub. No.: US 2026/0283551 A1
(43) Pub. Date: Sep. 24, 2026

(54) SENSOR ASSEMBLY WITH SPECTROPHOTOMETRIC SENSOR PORTION AND ELECTRODE SENSOR PORTION

(71) Applicant: EDWARDS LIFESCIENCES CORPORATION, Irvine, CA (US)

(72) Inventor: Matthew Dalene, Santa Ana, CA (US)

(21) Appl. No.: 19/478,312

(22) PCT Filed: Apr. 23, 2024
(86) PCT No.: PCT/US2024/025802

§ 371 Date: Oct. 24, 2025

Related U.S. Application Data
(60) Provisional application No. 63/497,906, filed on Apr. 24, 2023.

(51) Int. Cl. A61B 5/00; A61B 5/291; A61B 5/369

(52) U.S. Cl. CPC A61B 5/6814; A61B 5/0075; A61B 5/291; A61B 5/369; A61B 2562/066; A61B 2562/164

(57) ABSTRACT

A physiological sensor assembly is provided that includes a NIRS sensor portion, an electrode sensor portion, and a SA connector. The NIRS sensor portion has a light source, a light detector, and a flexible NS connector tail. The electrode sensor portion has first and second segments and a plurality of electrodes. The first segment has an electrode and a flexible FES connector tail. The second segment has an electrode and a flexible SES connector tail. The SA connector is in communication with the NIRS sensor portion, the electrode sensor portion first and second segments. The flexible NS connector tail, the flexible FES connector tail, and the flexible SES connector tail each extend outwardly from the SA connector independently of one another. The NIRS sensor portion, the electrode sensor portion first segment, and the electrode sensor portion second segment are independently positionable relative to one another.

### BACKGROUND OF THE INVENTION

### 1. Technical Field

The current application claims the benefit of and priority to U.S. Provisional Patent Application No. 63/497,906 entitled “Sensor Assembly with Spectrophotometric Sensor Portion and Electrode Sensor Portion” filed Apr. 24, 2023, the disclosure of which is hereby incorporated by reference in its entirety for all purposes. The present disclosure relates to medical devices that include use noninvasive oximetry sensors and electrodes.

### 2. Background Information

Near-infrared spectroscopy (NIRS) is an optical spectrophotometric method that can be used to continuously monitor tissue blood parameters such as oxygenation saturation. The NIRS method is based on the principle that light in the near-infrared range (700 nm to 1,000 nm) can pass easily through skin, bone and other tissues and can be detected thereafter. NIRS systems utilize one or more sensors that each include at least one light source and one or more light detectors for detecting reflected or transmitted light. The light signal is created and sensed in cooperation with a NIRS system that includes a processor and an algorithm for processing signals and the data contained therein. NIRS systems can be configured to determine cerebral oxygenation information. Such systems typically include sensors configured to be positioned on a patient's forehead; e.g., one sensor on each side of the patient's forehead to enable monitoring of each brain hemisphere.

Electroencephalography (“EEG”) is a non-invasive method used to sense and record electrical activity of the brain. EEG devices typically use one or more sensors configured to be disposed on a patient's skin surface. Each of these sensors include at least one electrode for sensing electrical activity. To measure cerebral electrical activity, it is desirable to position an EEG sensor on the patient's forehead and temple region.

In applications such as, but not limited to, estimating depth of anesthesia (DoA) (sometimes referred to as “depth of consciousness”) it is desirable to monitor both tissue oxygen saturation and cerebral electrical activity. In these applications, the need to position both NIRS sensors and EEG sensors on a patient's forehead can create sensor positioning issues with conventional sensors.

### SUMMARY

According to an aspect of the present disclosure, a physiological sensor assembly is provided that includes a near-infrared spectroscopy (NIRS) sensor portion, an electrode sensor portion, and a sensor assembly (SA) connector. The NIRS sensor portion has at least one light source, at least one light detector, and a flexible NIRS sensor (NS) connector tail. The electrode sensor portion has a first segment, a second segment, and a plurality of electrodes configured to sense electrical activity of a patient. The first segment has at least one electrode and a flexible first electrode segment (FES) connector tail. The second segment has at least one electrode and a flexible second electrode segment (SES) connector tail. The sensor assembly (SA) connector is in communication with the NIRS sensor portion, the electrode sensor portion first segment, and the electrode sensor portion second segment. The flexible NS connector tail, the flexible FES connector tail, and the flexible SES connector tail each extend outwardly from the SA connector independently of one another. The NIRS sensor portion, the electrode sensor portion first segment, and the electrode sensor portion second segment are independently positionable relative to one another.

In any of the aspects or embodiments described above and herein, the NIRS sensor assembly may include a first flex circuit that extends through the NS connector tail. The first flex circuit may have a plurality of first electrically conductive traces configured to provide electrical communication between the at least one light source and the SA connector and may be configured to provide electrical communication between the at least one light detector and the SA connector.

In any of the aspects or embodiments described above and herein, the electrode sensor portion may include a second flex circuit having a first segment portion that extends through the FES connector tail and a second segment portion that extends through the SES connector tail. The second flex circuit may have a plurality of second electrically conductive traces configured to provide electrical communication between the electrode sensor portion first segment and the SA connector and may be configured to provide electrical communication between the electrode sensor portion second segment and the SA connector.

In any of the aspects or embodiments described above and herein, the first flex circuit and the second flex circuit may be disposed in a stacked configuration and folded within the SA connector to form a folded portion.

In any of the aspects or embodiments described above and herein, the SA connector may include a stiffener panel having a bottom side surface, an opposing top side surface, and an insertion edge. The stiffener panel may be disposed within the folded portion with the first flex circuit and the second flex circuit disposed contiguous with the bottom side surface and the top side surface and extending around the insertion edge.

In any of the aspects or embodiments described above and herein, the NIRS sensor portion may have a body that includes a pad and houses the at least one light source and the at least one light detector. The body may be defined at least in part by a subject contact layer, a back cover, a plurality of lateral side surfaces, and a pair of end surfaces. The flexible NS connector tail may extend outwardly from the back cover at a position spaced apart from each end surface of the pair of end surfaces.

In any of the aspects or embodiments described above and herein, the flexible NS connector tail may be flexibly attached to the NIRS sensor body portion and may be positioned proximate a midpoint between the pair of end surfaces.

In any of the aspects or embodiments described above and herein, the electrode sensor portion first segment may have a body defined at least in part by a contact surface, an opposite rear surface, a plurality of lateral side surfaces, and a plurality of end surfaces. The flexible FES connector tail may extend outwardly from the rear surface at a middle position of the body.

In any of the aspects or embodiments described above and herein, the electrode sensor portion first segment may include a plurality of electrodes disposed along an axis, and the electrode sensor portion first segment may be configured for attachment to a patient forehead.

In any of the aspects or embodiments described above and herein, the electrode sensor portion second segment includes a single electrode, and the SES connector tail is configured to permit the electrode sensor portion second segment to be positioned for attachment to a patient temple area when the electrode sensor portion first segment is positioned for attachment to the patient forehead.

In any of the aspects or embodiments described above and herein, the electrode sensor portion may be configured as an electroencephalography (“EEG”) sensor.

According to an aspect of the present disclosure, a physiological sensor assembly is provided that includes a near-infrared spectroscopy (NIRS) sensor portion, an electrode sensor portion, and a sensor assembly (SA) connector. The near-infrared spectroscopy (NIRS) sensor portion has at least one light source, at least one light detector, a flexible NIRS sensor (NS) connector tail, and a first flex circuit. The at least one light source and the at least one light detector are in signal communication with the first flex circuit, and the first flex circuit extends throughout the NS connector tail. The electrode sensor portion has a plurality of electrodes configured to sense electrical activity of a patient, at least one flexible electrode sensor portion connector tail, and a second flex circuit, wherein the plurality of electrodes is in signal communication with the second flex circuit, and the second flex circuit extends throughout the at least one electrode sensor portion connector tail. The SA connector is in communication with the first flex circuit and the second flex circuit. The flexible NS connector tail and the at least one flexible electrode sensor portion connector tail each extend outwardly from the SA connector independently of one another. The first and second flex circuits are in a stacked and folded configuration within the SA connector.

In any of the aspects or embodiments described above and herein, the flexible NS connector tail and the at least one flexible electrode sensor portion connector tail may be independent of one another outside of the SA connector and the NIRS sensor portion and the electrode sensor portion may be independently positionable relative to one another.

In any of the aspects or embodiments described above and herein, the electrode sensor portion may include a first segment having at least one electrode, and a second segment having at least one electrode. The first and second segments may be independent of one another. The at least one flexible electrode sensor portion connector tail may include a flexible first electrode segment (FES) connector tail extending between the first segment and the SA connector, and a flexible second electrode segment (SES) connector tail extending between the second segment and the SA connector. The second flex circuit may include a first segment portion that extends through the FES connector tail and provides signal communication between the at least one electrode of the first segment and the SA connector, and a second segment portion that extends through the SES connector tail and provides signal communication between the at least one electrode of the second segment and the SA connector.

In any of the aspects or embodiments described above and herein, the first and second flex circuits may be disposed in a stacked configuration and folded within the SA connector to form a folded portion.

In any of the aspects or embodiments described above and herein, the SA connector may include a stiffener panel having a bottom side surface, an opposing top side surface, and an insertion edge. The stiffener panel may be disposed within the folded portion with the first flex circuit and the second flex circuit disposed contiguous with the bottom side surface and the top side surface and extending around the insertion edge.

According to an aspect of the present disclosure, a system for sensing a physiological parameter is provided that includes at least one physiological sensor assembly and a base unit. The at least one physiological sensor assembly includes a near-infrared spectroscopy (NIRS) sensor portion, an electrode sensor portion, and a sensor assembly (SA) connector. The NIRS sensor portion has at least one light source, at least one light detector, and a flexible NIRS sensor (NS) connector tail. The electrode sensor portion has a first segment, a second segment, and a plurality of electrodes configured to sense electrical activity of a patient. The first segment has at least one electrode and a flexible first electrode segment (FES) connector tail. The second segment has at least one electrode and a flexible second electrode segment (SES) connector tail. The SA connector is in communication with the NIRS sensor portion, and the electrode sensor portion first and second segments. The flexible NS connector tail, the flexible FES connector tail, and the flexible SES connector tail each extend outwardly from the SA connector independently of one another. The NIRS sensor portion, and the electrode sensor portion first and second segments are independently positionable relative to one another. The base unit has a system controller in communication with the NIRS sensor portion, the electrode sensor portion, and a non-transitory memory storing instructions. The instructions when executed cause the system controller to: a) control the NIRS sensor portion to control operation of the at least one light source and to receive signal communications from the at least one light detector; b) control the electrode sensor portion to sense the electrical activity of the patient using the plurality of electrodes; and c) process the signal communications from the at least one light detector and the sensed electrical activity of the patient to produce information relating to the physiological parameter.

In any of the aspects or embodiments described above and herein, the NIRS sensor assembly may include a first flex circuit that extends through the NS connector tail, the first flex circuit may have a plurality of first electrically conductive traces configured to provide electrical communication between the at least one light source and the SA connector, and may be configured to provide electrical communication between the at least one light detector and the SA connector.

In any of the aspects or embodiments described above and herein, the electrode sensor portion may include a second flex circuit having a first segment portion that extends through the FES connector tail and a second segment portion that extends through the SES connector tail. The second flex circuit may have a plurality of second electrically conductive traces configured to provide electrical communication between the electrode sensor portion first segment and the SA connector and may be configured to provide electrical communication between the electrode sensor portion second segment and the SA connector.

The foregoing features and elements may be combined in various combinations without exclusivity, unless expressly indicated otherwise. For example, aspects and/or embodiments of the present disclosure may include any one or more of the individual features or elements disclosed above and/or below alone or in any combination thereof. These features and elements as well as the operation thereof will become more apparent in light of the following description and the accompanying drawings. It should be understood, however, the following description and drawings are intended to be exemplary in nature and non-limiting.

### BRIEF DESCRIPTION OF THE DRAWINGS

FIG. 1 is a diagrammatic perspective view of a sensor assembly embodiment.

FIG. 2 is a diagrammatic planar view of a sensor assembly embodiment.

FIG. 3 is a diagrammatic view of a present disclosure system including a pair of present disclosure sensor assemblies.

FIG. 4 is an exploded diagrammatic perspective view of a portion of a NIRS sensor portion of a present disclosure sensor assembly.

FIG. 5 is a diagrammatic planar view of a portion of a NIRS sensor portion of a present disclosure sensor assembly from the attachment side.

FIG. 6 is a diagrammatic planar view of a portion of a NIRS sensor portion of a present disclosure sensor assembly from the rear side with a connector tail extending out from an end surface of the NIRS sensor body.

FIG. 7 is a diagrammatic planar view of a portion of a NIRS sensor portion of a present disclosure sensor assembly from the rear side with a connector tail extending out from a middle position of the NIRS sensor portion body.

FIG. 8 is a diagrammatic planar view of a portion of an electrode sensor portion segment of a present disclosure sensor assembly embodiment.

FIG. 9 is a diagrammatic sectional view of the electrode sensor portion shown in FIG. 8.

FIG. 10 is a diagrammatic planar view of a portion of an electrode sensor portion segment of a present disclosure sensor assembly embodiment.

FIG. 11 is a diagrammatic sectional view of the electrode sensor portion shown in FIG. 10.

FIG. 12 is a diagrammatic side view of a portion of an electrode sensor portion segment of a present disclosure sensor assembly embodiment.

FIG. 13 illustrates a pair of present disclosure sensor assemblies attached to a patient.

FIG. 14A diagrammatically illustrates a NIRS sensor portion flex circuit in an unfolded form.

FIG. 14B diagrammatically illustrates an electrode sensor portion flex circuit in an unfolded form.

FIG. 14C diagrammatically illustrates a NIRS sensor portion flex circuit and an electrode sensor portion flex circuit, both in unfolded form, in a stacked configuration.

FIGS. 14D and 14E diagrammatically illustrate a NIRS sensor portion flex circuit and an electrode sensor portion flex circuit in a stacked configuration, with the NIRS sensor portion flex circuit and the electrode sensor portion flex circuit in folded form.

FIG. 15 is a diagrammatic illustration of a NIRS flex circuit and an ES flex circuit partially folded about a fold axis and a stiffener panel.

FIG. 16 illustrates a present disclosure sensor assembly embodiment attached to a patient.

### DETAILED DESCRIPTION

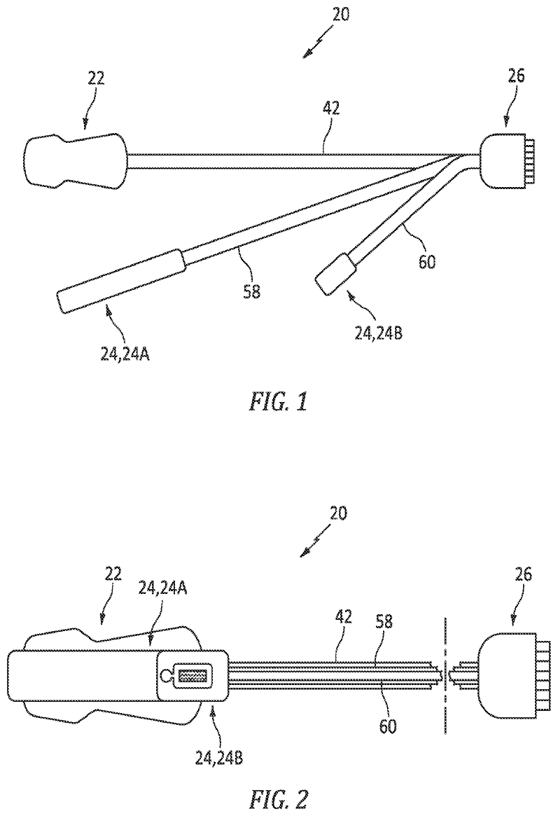

FIGS. 1 and 2 are diagrammatic views of a present disclosure sensor assembly 20 embodiment. The sensor assembly 20 includes a spectrophotometric sensor portion (referred to herein after as a NIRS sensor portion 22) and an electrode sensor portion 24. The NIRS sensor portion 22 and the electrode sensor portion 24 are connected to, and in signal communication with, a sensor assembly (SA) connector 26. FIG. 1 illustrates the NIRS sensor portion 22 and an electrode sensor portion 24 having two segments spread apart and FIG. 2 illustrates the same in a stacked configuration.

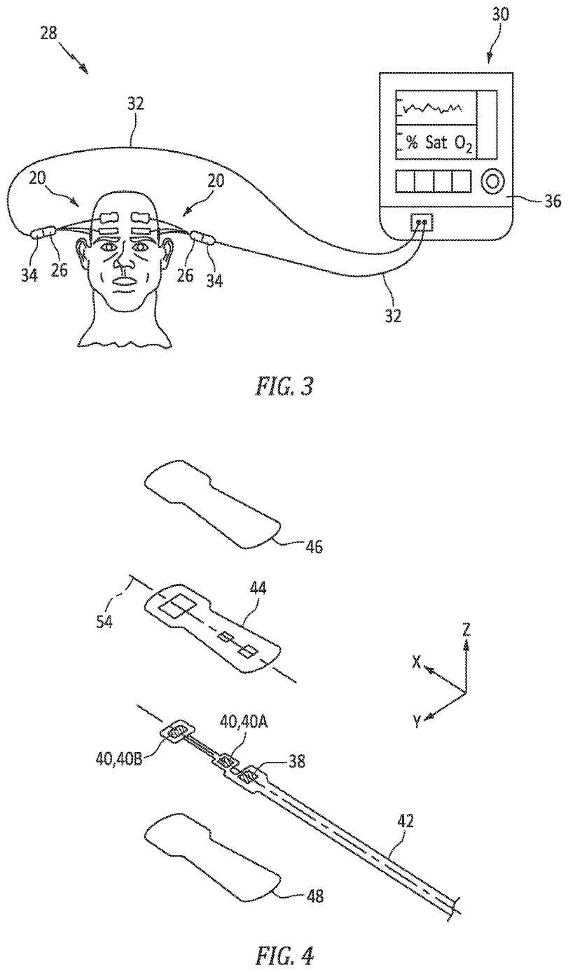

FIG. 3 diagrammatically illustrates a present disclosure patient monitoring system 28 embodiment. The system 28 includes a base unit 30, at least one sensor assembly 20 (FIG. 3 shows two (2) sensor assemblies 20), and one or more communication lines 32 that communicatively connect the sensor assembly(ies) 20 with the base unit 30. Each communication line (CL) 32 may include a CL connector 34 that mates with the SA connector 26. The communication lines 32 are operable to conduct signal data between sensor assemblies 20 and the base unit 30. In some embodiments, additional hardware including signal amplification devices may be in communication with the communications lines 32. The base unit 30 may include a display device, an input device, and a system controller 36. Examples of acceptable display devices include LED screens, LCD screens, and the like. Examples of acceptable input devices include a keyboard, a touch screen, a voice commanded unit, or the like. The present disclosure does not require communication lines 32 (e.g., independent of the sensor assembly 20) that communicatively connect the sensor assembly(ies) 20 with the base unit 30. For example, in some embodiments a sensor assembly 20 may be configured to be directly connected to a base unit 30, or a sensor assembly 20 may be configured for wireless communication with a base unit 30.

The system controller 36 is in communication with other system components including the sensor assemblies 20 and the like. The system controller 36 may be in communication with system components to control the operation of the respective component and/or to receive signals from and/or transmit signals to that component to perform the functions described herein. The system controller 36 may include any type of computing device, computational circuit, processor(s), CPU, computer, or the like capable of executing a series of instructions that are stored in memory. The instructions may include an operating system, and/or executable software modules such as program files, system data, buffers, drivers, utilities, and the like. The executable instructions may apply to any functionality described herein to enable the system 28 to accomplish the same algorithmically and/or coordination of system components. The system controller 36 includes or is in communication with one or more memory devices. The present disclosure is not limited to any particular type of memory device, and the memory device may store instructions and/or data in a non-transitory manner. Examples of memory devices that may be used include read-only memory, random access memory, volatile memory, non-volatile memory, static memory, dynamic memory, flash memory, cache memory, and/or any device that stores digital information. The system controller 36 may include, or may be in communication with, an input device that enables a user to enter data and/or instructions, and may include, or be in communication with, an output device configured, for example to display information (e.g., a visual display or a printer), or to transfer data, etc.

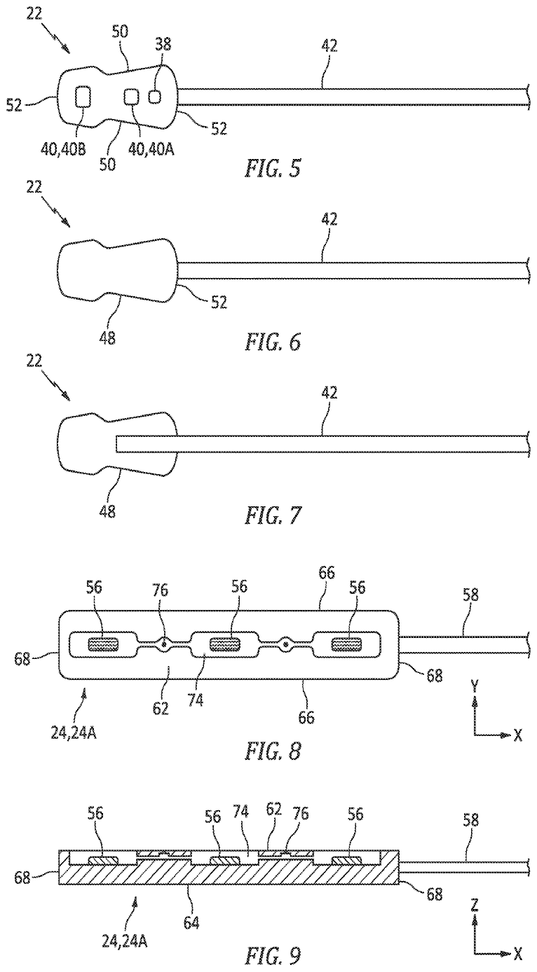

Referring to FIGS. 4-7, the present disclosure sensor assembly 20 is not limited to any particular NIRS sensor portion 22 configuration. A non-limiting example of a NIRS sensor portion 22 is shown in FIGS. 4 and 5. In this example, the NIRS sensor portion 22 includes one or more light sources 38, one or more light detectors 40, electrical circuitry that provides signal/electrical communication between the light sources and an SA connector 26, electrical circuitry that provides signal/electrical communication between the light detectors 40 and the SA connector 26, a pad 44, a subject contact layer 46, a back cover 48, and a NIRS sensor (NS) connector tail 42. The subject contact layer 46 is configured for contact with the patient's skin and the back cover 48 is opposite the subject contact layer 46. In some embodiments, a NIRS sensor portion 22 may have lateral side surfaces 50 and end surfaces 52 that extend (along the Z-axis) between the contact layer 46 and the back cover 48. The NIRS sensor portion 22 may be described as having a body defined at least in part by the subject contact layer 46, the back cover 48, the lateral side surfaces 50, and the end surfaces 52. The NIRS sensor portion 22 has a lengthwise extending longitudinal axis 54 (e.g., extending along the X-axis) and a widthwise extending axis (e.g., extending along the Y-axis). The light source 38 and light detectors 40 are spaced apart from one another along the lengthwise axis 54. The NIRS sensor portion 22 body is flexible enough to conform with a variety of different patient skin surface geometries.

The light source(s) 38 may include one or more light emitting components (e.g., light emitting diodes (LEDs), laser diodes, etc.) that are selectively operable to produce infrared light at one or more predetermined wavelengths (i.e., light in the range of about seven hundred nanometers (700 nm) to about one thousand nanometers (1,000 nm)). In some embodiments, a light source 38 may also be configured to produce visible light (i.e., light in the wavelength range of about three hundred ninety nanometers (390 nm) to about seven hundred fifty nanometers (750 nm)). The light source 38 is in electrical/signal communication with the SA connector 26. A flexible electrical circuit (“flex circuit”) is a nonlimiting example of electrical circuitry for providing the aforesaid electrical communication. In some embodiments, the flex circuit may extend through the NS connector tail 42 and be in electrical communication with the SA connector 26. In another nonlimiting example, the light source(s) 38 may be in communication with a printed circuit board (PCB) and that PCB may be in communication with the SA connector 26 by wired connection.

The light detectors 40 include one or more light responsive transducers (e.g., photodiodes, charge-coupled devices etc.) that are operable to detect light emitted by the light source 38 after such light passes through a portion of the patient's body. The light detectors 40 are in electrical/signal communication with the SA connector 26. In those embodiments wherein the NIRS sensor portion 22 includes more than one light detector 40, the light detector 40 closest to the light source 38 may be referred to hereinafter as the “near detector 40A”, and the light detector 40 farthest from the light source 38 may be referred to hereinafter as the “far detector 40B”. The relative positioning of the light source 38 and the light detectors 40A, 40B on the NIRS sensor portion 22 can be varied for different applications; e.g., to distinguish scalp tissue and skull tissue from brain tissue, etc. The NIRS sensor portion 22 is not limited to any particular positioning of the light source 38 and the light detectors 40A, 40B. U.S. Pat. No. 10,321,862, which is hereby incorporated by reference in its entirety, discloses several examples of acceptable light source 38/light detector 40 relative positioning. A flexible electrical circuit (“flex circuit”) is a nonlimiting example of electrical circuitry for providing electrical communication between the light detectors 40 and the SA connector 26. In another nonlimiting example, the light detectors 40 may be in communication with a printed circuit board (PCB) and that PCB may be in communication with the SA connector 26 by wired connection.

The NIRS sensor portion 22 may include components in addition to the light source(s), light detector(s), and the electrical/signal circuitry (e.g., flex circuit). For example, a NIRS sensor portion 22 may include one or more electrical insulating layers, one of more EMI shielding layers, and the like, and any combination thereof. In some embodiments, an adhesive layer may be disposed on the subject contact layer 46 to facilitate the NIRS sensor portion 22 being attached to the patient's skin surface. In those embodiments wherein the NIRS sensor portion 22 includes an adhesive layer, the NIRS sensor portion 22 may include a removable protective layer disposed on the exposed surface of the adhesive layer to protect it before the NIRS sensor portion 22 is applied.

Referring to FIGS. 5-7, the NS connector tail 42 may extend outwardly from an end surface 52 (e.g., the end surface 52 proximate the light source 38-see FIGS. 5 and 6), or from a lateral side surface 50, or from the back cover 48 at a position between the end surfaces 52; e.g., approximately at a mid-point of the body (see FIG. 7). The flexible NS connector tail 42 may be flexible at the back cover 48 point of attachment. It has been discovered that a NIRS sensor body having a NS connector tail 42 that extends out from the back cover 48 at a position between the end surfaces 52 is less apt to pull away from the subject's skin surface in the event tension is applied to the NS connector tail 42.

The present disclosure is not limited to any particular NIRS sensor portion 22 configuration. U.S. Pat. Nos. 9,888,873; 10,321,862; 10,786,157; and 10,881,337, and U.S. provisional Patent Application No. 63,482,279 filed Jan. 30, 2023, each of which is hereby incorporated by reference in its entirety, disclose examples of NIRS sensors that may be modified for use as a NIRS sensor portion 22 as described herein in their respective entirety or in relevant portion.

The electrode sensor portion 24 includes one or more electrodes 56 for sensing the electrical activity of a patient. The present disclosure is described herein in terms of an electrode sensor portion 24 configured to function as an electroencephalograph (EEG) sensor. The present disclosure is not, however, limited to an electrode sensor portion 24 configured for EEG sensing purposes.

The electrode sensor portion 24 may be configured as a single body or may include a plurality of segments. The present disclosure is not limited to any electrode sensor portion 24 configuration. In those embodiments wherein an electrode sensor portion 24 is a single body, the electrode sensor portion 24 may include an electrode sensor connector tail in electrical communication with the SA connector 26. As detailed herein, present disclosure embodiments may provide communication between a sensor portion 24 and an SA connector 26 in a variety of different ways; e.g., by flexible circuit, by PCB, by PCB and wired connection, and the like. In the embodiment shown in FIGS. 1 and 2, the electrode sensor portion 24 includes a first segment 24A in electrical communication with the SA connector 26 via a first electrode segment (FES) connector tail 58 and a second segment 24B in electrical communication with the SA connector 26 via a second electrode segment (SES) connector tail 60. The FES connector tail 58 and the SES connector tail 60 permit variable positioning between the first and second electrode segments 24A, 24B to suit the application; e.g., the first electrode segment 24A may be applied to the patient's forehead and the second electrode segment 24B may be applied to the patient's temple region. The electrode sensor portion 24 (i.e., single body or segments) configurations are conformable to a variety of different skin surface geometries.

The first electrode segment 24A may have a rectangular geometric configuration as shown in FIGS. 1, 2, 8, and 9 but is not limited to that geometric configuration. The first electrode segment example shown in FIGS. 1, 2, 8, and 9 includes a body defined by a contact surface 62, an opposite rear surface 64, lateral side surfaces 66, and end surfaces 68. The lateral side surfaces 66 (e.g., extending along an X-axis) are on opposite lateral sides of the first electrode segment 24A, and the end surfaces 68 (e.g., extending along a Y-axis) are on opposite ends of the first electrode segment 24A. In the example embodiment shown in the FIGS. 1, 2, 8, and 9, the lateral side surfaces 66 and the end surfaces extend (e.g., in a Z-axis direction) between the contact surface 62 and rear surface 64. The first electrode segment 24A may be described as having a longitudinal axis that extends between the end surfaces 68, a widthwise axis that extends between the lateral side surfaces 66, and a height (or “thickness”) that extends between the contact surface 62 and the rear surface 64.

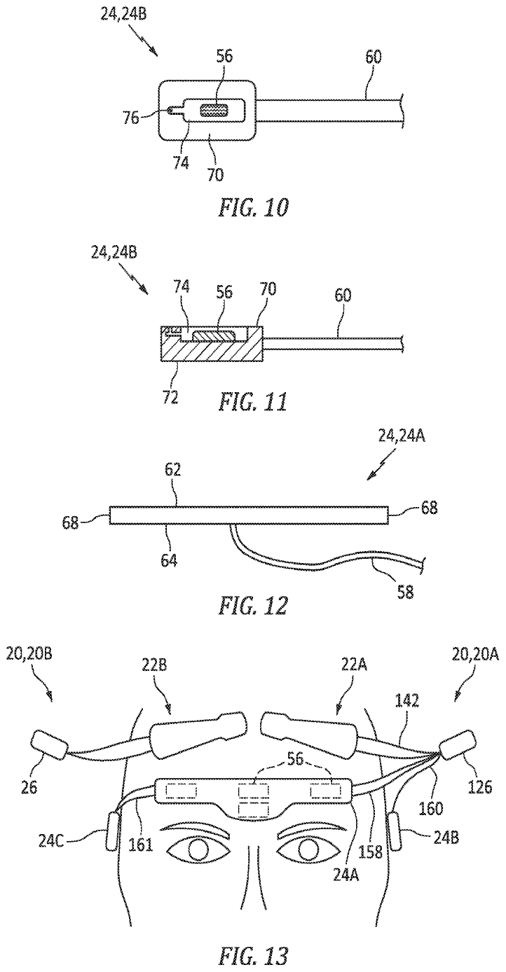

The second electrode segment 24B may have a rectangular geometric configuration as shown in FIGS. 10 and 11 but is not limited to that geometric configuration. The second electrode segment example shown in FIGS. 10 and 11 includes a body defined by a contact surface 70, an opposite rear surface 72, and lateral side surfaces that extend between the contact surface 70 and the rear surface 72.

Referring to FIGS. 7-12, the FES and/or SES connector tail 58, 60 may extend outwardly from an end surface, or from a lateral side surface, or from the respective rear surface of the electrode segment 24A, 24B. In those embodiments wherein the FES and/or SES connector tail 58, 60 extends outwardly from the rear surface of an electrode segment 24A, 24B, the point of attachment may be located approximately in the middle of the electrode portion segment 24A, 24B (see FIG. 12). The flexible FES/SES connector tail 58, 60 may be flexible at the rear surface point of attachment. It has been discovered that an electrode segment body having a connector tail 58, 60 that extends out from the rear surface at a middle position is less apt to pull away from the subject's skin surface in the event tension is applied to the connector tail 58, 60.

In the embodiment shown in FIGS. 8-11, the first electrode segment 24A is shown including three electrodes 56 and the second electrode segment 24B including a single electrode 56. The present disclosure is not limited to these electrode 56 configurations; e.g., the first electrode segment 24A may include fewer than three electrodes 56 or more than three electrodes 56 and the second electrode segment 24B may include more than one electrode 56.

Each electrode 56 may be disposed within a respective pocket 74 formed in the respective first and second electrode segments 24A, 24B. The pockets 74 are open to the respective contact surface. Typically, a pocket 74 is configured so that the sensing surface of the electrode 56 is disposed below the contact surface leaving room for electrolytic gel between the electrode sensing surface and the contact surface.

In the embodiment shown in FIGS. 8 and 9, the electrodes 56 in the first electrode segment are aligned along a lengthwise-extending longitudinal axis extending between the first and second end surfaces 68. In alternative embodiments, the electrodes 56 may not be aligned on a common axis. Electrode sensor portion 24 embodiments may be configured so an electrode 56 operates as a reference electrode, and other electrodes 56 each operate as an active channel electrode. The present disclosure is not limited to any particular operational electrode 56 configuration.

As stated herein, the electrode sensor portion 24 may assume a variety of different configurations. FIG. 13 illustrates a first sensor assembly 20A and a second sensor assembly 20B. The first sensor assembly 20A has a first NIRS sensor portion 22A, a first electrode sensor portion segment 24A, a second electrode sensor portion segment 24B, and a third electrode sensor portion segment 24C. The first NIRS sensor portion 22A is in signal communication with the first SA connector 126 via a first NS connector tail 142. The first electrode sensor portion 24A is in signal communication with the first SA connector 126 via a first FES connector tail 158. The second segment 24B is in signal communication with the first SA connector 126 via a SES connector tail 160. The third segment 24C is in signal the connector 126 via a third electrode segment (TES) connector tail 161 that passes through the first segment 24A. In this embodiment, the electrode sensor portion first segment 24A includes a plurality of electrodes 56 (four (4) electrodes are shown) and is configured to span the patient's forehead. The second and third segments 24B, 24C are configured for placement on the patient's left and right temple regions. The second sensor assembly 20B has a second NIRS sensor portion 22B attached to the opposite side of the patient's forehead. This example illustrates two alternative present disclosure sensor assembly 20 configurations to illustrate non-limiting examples of present disclosure sensor assembly 20 configurations.

The first and second electrode segments 24A, 24B of the electrode sensor portion 24 may be configured in a variety of ways. For example, the aforesaid segments 24A, 24B may be a unitary body formed from a single material or may be formed from a plurality of layers that collectively form the respective segment 24A, 24B. In those embodiments that include a unitary body, the body may be configured to contain electrical circuitry, including for example communication lines (e.g., a copper or gold wires) extending from the respective electrodes 56, or a flexible circuit/printed circuit board (PCB) in communication with the electrodes 56 (detailed below), or any combination thereof. In those embodiments having a segment 24A, 24B that includes a plurality of layers, the electrical communication lines or flexible circuit/PCB may form, or be disposed in a layer, or be disposed between layers of the segment 24A, 24B. In some embodiments, the electrodes 56 (and any other elements) included in a segment 24A, 24B may be in communication with a flexible circuit that extends through the connector tail 58, 60 and is in communication with the SA connector 26, 126. In some embodiments, the electrodes 56 (and any other elements) included in a segment 24A, 24B may be in communication with a flexible circuit/PCB disposed within the respective segment 24A, 24B, 24C, and that flexible circuit/PCB may be in communication via wires with the SA connector 26, 126. In some embodiments, the first electrode segment 24A may be configured differently from the second electrode segment 24B (e.g., different materials, layers, and the like) or both segments 24A, 24B may have the same configuration.

The segments 24A, 24B may comprise one or more generally flexible materials (e.g., polymeric materials such as a polyimide). The degree to which a segment 24A, 24B is flexible can vary depending on the intended application of the sensor assembly 20; e.g., sufficiently flexible to readily correspond to curvature of the application site, etc. In some embodiments, a segment 24A, 24B may comprise a material that is breathable (air and/or moisture) to increase comfort and to facilitate retention of the segment on the patient's skin.

In some embodiments, the electrode sensor portion segments 24A, 24B may be configured for attachment to the patient's skin surface via a layer of adhesive disposed on a respective contact surface. The present disclosure is not limited to using adhesive for maintaining contact between the respective segment 24A, 24B and the patient's skin surface. In alternative embodiments, the respective segments 24A, 24B may be configured to maintain contact with the patient's skin surface via an independent element such as an elastic member, a bandage member, a strap, a cap, any combination of the same, or other devices for fastening sensors to a patient's body or skin known in the art. The present disclosure is not limited to any particular configuration for maintaining contact between the respective electrode sensor portion segment 24A, 24B and the patient's skin surface.

In some embodiments, the segments 24A, 24B may each include a removable protection layer that is initially disposed in contact with the respective contact surface but is intended is to be removed prior to use. The removable protection layer may be adhered to the respective contact surface using an adhesive.

The present disclosure sensor assemblies 20 may include an electrolytic gel disposed in communication with the sensing surface of each electrode 56. The electrolytic gel is configured to facilitate electrical signal transmission from patient to the electrode 56; e.g., to improve electrical signal conduction between the patient and the sensing surface of the electrode 56. The present disclosure is not limited to any particular type of electrolytic gel. In some embodiments a sensor assembly 20 may include a porous medium such as an open cell foam (e.g., a reticulated foam) disposed within a pocket 74. The porous medium may be disposed in the region of the pocket 74 between the electrode 56 sensing surface and the plane across the opening of the pocket 74 that is coplanar with the contact surface. The porous medium may facilitate retention of the electrolytic gel within the pocket 74.

In some embodiments (e.g., see FIGS. 8-11), one or both of the electrode sensor portion 24 segments may include electrolytic gel containment features including pockets 74 configured to retain electrolytic gel (e.g., pockets 74 configured to provide a void around at least a portion of the periphery of the electrode 56 disposed in the pocket 74) and a gel vent 76 disposed in fluid communication with the respective pocket 74. The gel vent 76 may include at least one vent passage that extends between the gel vent 76 and an exterior surface of the segment 24A, 24B. If a force is applied on or near the pocket 74 that decreases the volume of the pocket 74, gel may exit the pocket 74 and enter the gel vent 76. Conversely, if the force that decreased the volume of the pocket 74 is no longer applied and the pocket 74 elastically returns to its initial volume, gel may exit the gel vent 76 and reenter the pocket 74.

Embodiments of the present disclosure sensor assembly 20 are configured to permit modular construction. The term “modular construction” is used here to refer to the sensor assembly 20 being configured in a manner that allows the sensor to be assembled with a NIRS sensor portion 22 and an electrode sensor portion 24, or just a NIRS sensor portion 22, or just an electrode sensor portion 24. In all of these configurations, the SA connector 26 is assembled in like manner and may be connectable to the same mating connector; e.g., a CL connector 34. The aforesaid configuration of the present disclosure sensor assembly 20 that permits modular construction is understood to be significant for a variety of different reasons. For example, the modularity permits a number of different sensor assembly 20 configurations to be manufactured in substantially the same manner; e.g., components are included or not based on the particular sensor assembly configuration being produced. This approach utilizes common components for different sensor assembly 20 configurations, common tooling for producing different sensor assembly 20 configurations, common technician training for different sensor assembly 20 configurations, and the like. In this manner, the modularity is understood to provide significant benefits in sensor assembly 20 manufacturing. The modularity and the common components utilized are also understood to provide significant supply chain benefits. Embodiments of the present disclosure sensor assembly 20 may be configured to be separately identifiable by the base unit 30; e.g., once the sensor assembly 20 is in signal communication with the base unit 30, the sensor assembly 20 configuration is identifiable as having a NIRS sensor portion 22 and an electrode sensor portion 24, or as having only a NIRS sensor portion 22, or as having only an electrode sensor portion 24. In this manner, the present disclosure sensor assembly 20 provides greater utility to the end user; e.g., the end user can use the specific configuration desired. For example, in certain circumstances the end user may only need to collect data provided by a NIRS sensor portion 22, or only data provided by an electrode sensor portion 24, but not both. In those instances, a NIRS sensor portion sensor assembly 20 configuration can be used, or an electrode sensor portion sensor assembly 20 configuration can be used rather than a NIRS sensor portion 22 plus electrode sensor portion 24 configuration, and a consequent cost savings can be achieved.

As indicated herein, the NIRS sensor portion 22 of the present disclosure sensor assembly 20 may include one or more light sources 38, one or more light detectors 40, electrical circuitry that provides signal/electrical communication between the light sources 38 and the SA connector 26, electrical circuitry that provides signal/electrical communication between the light detectors 38 and the SA connector 26, and an NS connector tail 42. The electrical circuitry that provides signal/electrical communication between the light sources 38 and the SA connector 26, and between the light detectors 40 and the SA connector 26 may take the form of a flexible electrical circuit (“NIRS flex circuit 78”) and that NIRS flex circuit 78 may extend through the NS connector tail 42 and be in electrical communication with the SA connector 26. In some embodiments, the NIRS flex circuit 78 may include a plurality of layers, including one or more of electrical trace layers, electrically insulative layers, electromagnetic interference (“EMI”) shield layers, and top and bottom cover layers.

The electrical trace layers may include electrically conductive members (i.e., “traces” or “wires”), some of which are configured to conduct electrical signals relative to electrical components within the NIRS sensor (e.g., the light source(s), the light detector(s), and the like) and some of which are configured to provide EMI shielding. The specific number of traces within the respective electrical trace layers can vary depending on the number of electrical components disposed within the NIRS sensor portion 22. The traces may be formed from an electrically conductive material such as copper, or an electrically conductive polymer, or the like, or may be formed from a first material that is coated with a second material that is electrically conductive, or the like. The term “electrically conductive” as used herein describes a material that is adequate to conduct electrical signals of the type and power necessary to conduct electrical signals to and from components within the NIRS sensor portion 22 with an acceptably low level of resistance and/or interference. All of the respective layers within the NIRS flex circuit 78 may be stacked to produce communication between traces where desired, electrical insulation where desired, and EMI shielding where desired.

The top and bottom cover layers may be formed of an electrically insulative material (e.g., a polyamide polymer film (e.g., Kapton® polyamide film produced by E.I. du Pont de Nemours and Company) that both insulates and protects the NIRS flex circuit 78.

U.S. Pat. No. 10,881,337 discloses a non-limiting example of a flex circuit that may be used in part within the present disclosure. The present disclosure may be used with different flex circuit configurations and is not therefore limited to the flex circuit configuration disclosed within U.S. Pat. No. 10,881,337.

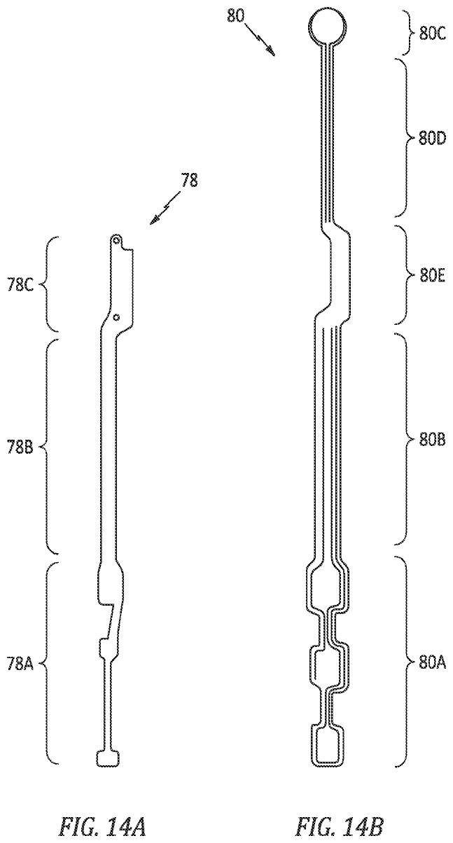

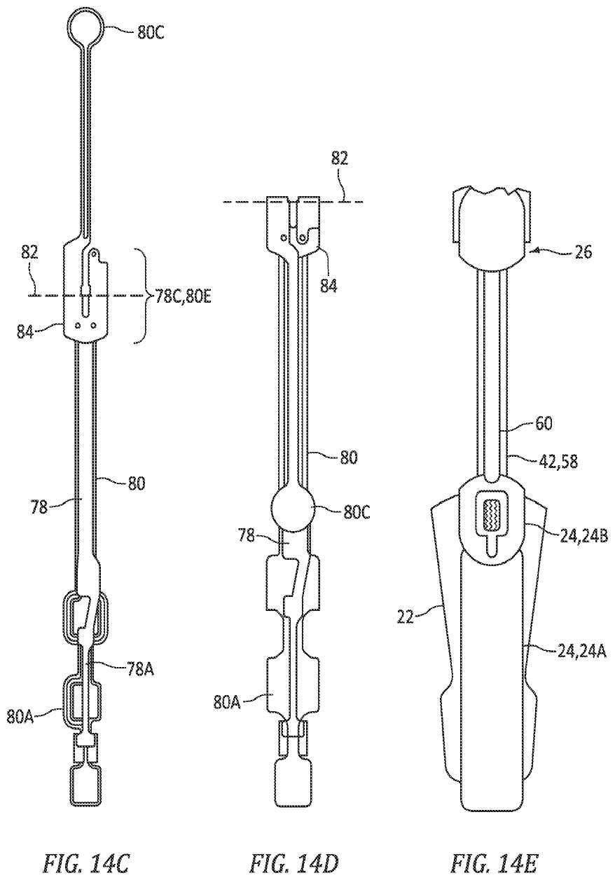

FIG. 14A diagrammatically illustrates a NIRS flex circuit 78 in an unfolded form (as will be detailed below). FIG. 14A diagrammatically shows only the flex circuit portion and does not show the pad and other elements that are included in the body of the NIRS sensor portion 22 to facilitate the description herein. The sensor body portion 78A, connector tail portion 78B, and the connector portion 78C of the NIRS flex circuit 78 are identified in FIG. 14A.

The electrode sensor portion 24 includes one or more electrodes 56 for sensing the electrical activity of a patient. The electrode sensor portion 24 may include electrical circuitry that provides signal/electrical communication between the electrodes and the connector, and the electrical circuitry may take the form of an electrode sensor(ES) flex circuit 80 that extends through a connector tail and is in electrical communication with the SA connector 26. The ES flex circuit 80 may include a plurality of layers, including one or more of electrical trace layers, electrically insulative layers, electromagnetic interference (“EMI”) shield layers, and top and bottom cover layers. FIG. 14B diagrammatically illustrates an ES flex circuit 80 in an unfolded form (as will be detailed below). The ES flex circuit shown in FIG. 14B is configured for an electrode sensor portion 24 that has first and second electrode segments 24A, 24B. The first electrode segment portion 80A, the FES connector tail portion 80B, the second electrode segment portion 80C, the SES connector tail portion 80D, and the connector portion 80E of the ES flex circuit 80 are identified in FIG. 14B. FIG. 14B diagrammatically shows only the flex circuit portion 80 and does not show the electrode sensor portion pad or other elements that may be included in the body of the electrode sensor portion 24 to facilitate the description herein.

FIG. 14C diagrammatically illustrates the NIRS flex circuit 78 and the ES flex circuit 80, both in unfolded form, in a stacked configuration. FIG. 14C illustrates a fold axis 82 disposed in the connector portions 78C, 80E of the stacked NIRS flex circuit 78 and ES flex circuit 80.

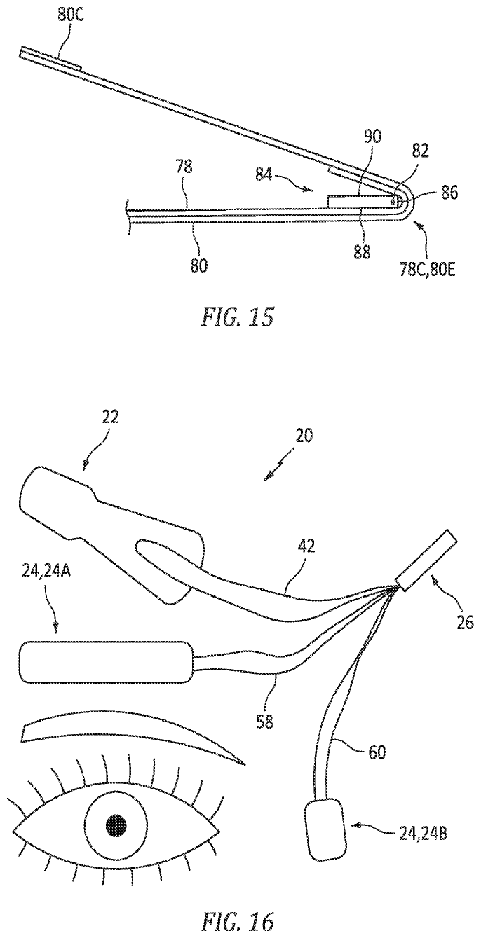

Referring to FIGS. 14C, 14D, and 15, in some embodiments a stiffener panel 84 may be disposed relative to the fold axis 82. The stiffener panel 84 is a relatively rigid component that provides structural rigidity to the SA connector 26. The stiffener panel 84 includes a plurality of edges (including an insertion edge 86) that extend between a bottom side surface 88 and an opposing top side surface 90. The stiffener panel 84 may be made of various different materials or combinations of materials; e.g., a polymeric material. The rigidity provided by the stiffener panel 84 makes it easier for a user to insert the SA connector 26 into, or remove the SA connector 26 from, a mating connector element.

FIGS. 14D and 14E illustrate the NIRS flex circuit 78 and the ES flex circuit 80 in a stacked configuration, with the NIRS flex circuit 78 and the ES flex circuit 80 folded about the fold axis 82. FIG. 15 shows the NIRS flex circuit 78 and the ES flex circuit 80 partially folded about the fold axis 82 (and the stiffener panel 84). In the folded form, electrical traces from the respective electrical trace layers of the NIRS flex circuit 78 and the ES flex circuit 80 are exposed at the SA connector 26 distal end and positioned for communication with electrical traces disposed within a mating connector. In those embodiments that include a stiffener panel 84, the connector portions 78C, 80E of the respective flex circuits 78, 80 may be folded around the stiffener panel 84, thereby capturing the stiffener panel 84 therebetween. In some embodiments, external housing members may be utilized to retain a portion of the folded flex circuits 78, 80, and define the exposed portions of the flex circuits 78,80.

As can be seen in FIGS. 1, 2, and 14, the present disclosure sensor assembly 20 is configured to permit considerable configuration flexibility regarding where the NIRS sensor portion 22 and the electrode sensor portion 24 segments can be positioned on the patient. More specifically, the flexible NS connector tail 42, FES connector tail 58, and SES connector tail 60 are all cantilevered out, and flexible after exiting, from the SA connector 26 and provide the end user with an enhanced ability to position the respective NIRS sensor portion 22, and the first and second electrode segments 24A, 24B of the electrode sensor portion 24 where desired; i.e., positional degrees of freedom between the NIRS sensor portion 22 and the electrode sensor portion 24. The flexible NS connector tail 42, the flexible FES connector tail 58, and the flexible SES connector tail 60 each extend outwardly from the SA connector 26 independently of one another, and each is sufficiently flexible to make the NIRS sensor portion 22, the electrode sensor portion first segment 24A, and the electrode sensor portion second segment 24B independently positionable relative to one another. In other words, only the length of the respective connector tails 42, 58, 60 and their connection to the SA connector 26 limit the relative positioning of the NIRS sensor portion 22 and the electrode sensor portion first and second segments 24A, 24B. A person of skill in the art will recognize that patient “features” (e.g., hairlines, forehead contour, skin bruising, and the like) will often influence where an end-user will wish to apply a NIRS sensor portion 22 and/or an electrode sensor portion 24. The present disclosure sensor assembly 20 permits the user to place the NIRS sensor portion 22 and/or an electrode sensor portion 24 in desirable positions and to avoid problematic positions.

Furthermore, the description above is directed to a present disclosure sensor assembly 20 having a NIRS sensor portion 22 and an electrode sensor portion 24. As stated above, however, alternative embodiments of the present disclosure sensor assembly 20 may only include a NIRS sensor portion 22 or an electrode sensor portion 24. In these embodiments, the flexible connector tails 42, 58, 60 also provide the end-user with an enhanced ability to position the respective sensor portion where desired.

During use of the present disclosure system and/or sensor assemblies, an end-user (e.g., physician, nurse, etc.) may remove a sensor assembly 20 from its packaging and determine which side of the patient's forehead the user wishes to apply the sensor assembly 20. Based on that determination, the user can then arrange the electrode sensor portion(s) 24 relative to the NIRS sensor portion 22. Once an appropriate arrangement is determined, the end-user may apply the electrode sensor portion first segment 24A to the patient's forehead above the eyebrow and the electrode segment portion second segment 24B to the patient's temple region on that side of the forehead. The user may then apply the NIRS sensor portion 22 to the patient's forehead above the electrode sensor portion first segment 24A. The same process can be followed for a second present disclosure sensor assembly 20 on the opposite side of the patient's forehead. In this manner, the pair of sensor assemblies 20 can be used to collect data from each hemisphere of the patient's brain. The sensor assemblies 20 can be connected to the base unit 30 of the patient monitoring system 28 by connecting the communication lines 32 from the base unit 30 to the SA connector 26 of each respective sensor assembly 20. During operation of the patient monitoring system 28, stored instructions accessible by the system controller 36 can be used to control the operation of the NIRS sensor portion light source(s) 38 and can be used to receive and process signal communications from the NIRS sensor portion 22 and electrode sensor portion 24 of each sensor assembly 20. The signals representative of the electrical activity sensed from the patient can be processed according to known algorithms for producing EEG data. The signals representative of light detected by the light detectors 40 of the NIRS sensor portions 22 (i.e., light from the respective light source 38 that has passed through patient tissue) can be processed according to known algorithms and methodologies for producing tissue oxygen saturation data and the like. Nonlimiting examples of algorithms and methodologies for producing tissue oxygen saturation data and the like are disclosed in U.S. Pat. Nos. 7,072,701; 8,078,250; 8,428,674; 8,788,004; 8,897,848; 8,923,943; 8,965,472; 9,456,773; 9,848,808; 10,117,610 10,261,010; 10,485,462; 10,918,321; and 11,454,589, each of which is hereby incorporated by reference in its entirety. These issued patents, commonly assigned with the present application, represent non-limiting examples of algorithms and methods that may be utilized with the present disclosure.

While the principles of the disclosure have been described above in connection with specific apparatuses and methods, it is to be clearly understood that this description is made only by way of example and not as limitation on the scope of the disclosure. Specific details are given in the above description to provide a thorough understanding of the embodiments. However, it is understood that the embodiments may be practiced without these specific details.

The singular forms “a,” “an,” and “the” refer to one or more than one, unless the context clearly dictates otherwise. For example, the term “comprising a sample” includes single or plural samples and is considered equivalent to the phrase “comprising at least one sample.” The term “or” refers to a single element of stated alternative elements or a combination of two or more elements unless the context clearly indicates otherwise. As used herein, “comprises” means “includes.” Thus, “comprising A or B,” means “including A or B, or A and B,” without excluding additional elements.

It is noted that various connections are set forth between elements in the present description and drawings (the contents of which are included in this disclosure by way of reference). It is noted that these connections are general and, unless specified otherwise, may be direct or indirect and that this specification is not intended to be limiting in this respect. Any reference to attached, fixed, connected or the like may include permanent, removable, temporary, partial, full and/or any other possible attachment option.

No element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprise”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

While various inventive aspects, concepts and features of the disclosures may be described and illustrated herein as embodied in combination in the exemplary embodiments, these various aspects, concepts, and features may be used in many alternative embodiments, either individually or in various combinations and sub-combinations thereof. Unless expressly excluded herein all such combinations and sub-combinations are intended to be within the scope of the present application. Still further, while various alternative embodiments as to the various aspects, concepts, and features of the disclosures-such as alternative materials, structures, configurations, methods, devices, and components, and so on-may be described herein, such descriptions are not intended to be a complete or exhaustive list of available alternative embodiments, whether presently known or later developed. Those skilled in the art may readily adopt one or more of the inventive aspects, concepts, or features into additional embodiments and uses within the scope of the present application even if such embodiments are not expressly disclosed herein. For example, in the exemplary embodiments described above within the Detailed Description portion of the present specification, elements may be described as individual units and shown as independent of one another to facilitate the description. In alternative embodiments, such elements may be configured as combined elements. It is further noted that various method or process steps for embodiments of the present disclosure are described herein. The description may present method and/or process steps as a particular sequence. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the description should not be construed as a limitation.

Additionally, even though some features, concepts, or aspects of the disclosures may be described herein as being a preferred arrangement or method, such description is not intended to suggest that such feature is required or necessary unless expressly so stated. Still further, exemplary, or representative values and ranges may be included to assist in understanding the present application, however, such values and ranges are not to be construed in a limiting sense and are intended to be critical values or ranges only if so expressly stated.

The treatment techniques, methods, and steps described or suggested herein or in references incorporated herein may be performed on a living animal or on a non-living simulation, such as on a cadaver, cadaver heart, anthropomorphic ghost, or simulator (e.g., with the body parts, or tissue being simulated).

Any of the various systems, devices, apparatuses, etc. in this disclosure may be sterilized (e.g., with heat, radiation, ethylene oxide, hydrogen peroxide) to ensure they are safe for use with patients, and the methods herein may comprise sterilization of the associated system, device, apparatus, etc. ; e.g., with heat, radiation, ethylene oxide, hydrogen peroxide.

1. A physiological sensor assembly, comprising:
a near-infrared spectroscopy (NIRS) sensor portion having at least one light source, at least one light detector, and a flexible NIRS sensor (NS) connector tail; and
an electrode sensor portion having a first segment, a second segment, and a plurality of electrodes configured to sense electrical activity of a patient, the first segment having at least one said electrode and a flexible first electrode segment (FES) connector tail, the second segment having at least one said electrode and a flexible second electrode segment (SES) connector tail; and
a sensor assembly (SA) connector in communication with the NIRS sensor portion, the electrode sensor portion first segment, and the electrode sensor portion second segment;
wherein the flexible NS connector tail, the flexible FES connector tail, and the flexible SES connector tail each extend outwardly from the SA connector independently of one another, and wherein the NIRS sensor portion, the electrode sensor portion first segment, and the electrode sensor portion second segment are independently positionable relative to one another.

2. The sensor assembly of claim 1, wherein the NIRS sensor assembly includes a first flex circuit that extends through the NS connector tail, the first flex circuit having a plurality of first electrically conductive traces configured to provide electrical communication between the at least one light source and the SA connector, and configured to provide electrical communication between the at least one light detector and the SA connector.

3. The sensor assembly of claim 2, wherein the electrode sensor portion includes a second flex circuit having a first segment portion that extends through the FES connector tail and a second segment portion that extends through the SES connector tail, the second flex circuit having a plurality of second electrically conductive traces configured to provide electrical communication between the electrode sensor portion first segment and the SA connector, and configured to provide electrical communication between the electrode sensor portion second segment and the SA connector.

4. The sensor assembly of claim 3, wherein the first flex circuit and the second flex circuit are disposed in a stacked configuration and folded within the SA connector to form a folded portion.

5. The sensor assembly of claim 4, wherein the SA connector includes a stiffener panel having a bottom side surface, an opposing top side surface, and an insertion edge;
wherein the stiffener panel is disposed within the folded portion with the first flex circuit and the second flex circuit disposed contiguous with the bottom side surface and the top side surface and extending around the insertion edge.

6. The sensor assembly of claim 1, wherein the NIRS sensor portion has a body that includes a pad and houses the at least one light source and the at least one light detector, wherein the body is defined at least in part by a subject contact layer, a back cover, a plurality of lateral side surfaces, and a pair of end surfaces;
wherein the flexible NS connector tail extends outwardly from the back cover at a position spaced apart from each end surface of the pair of end surfaces.

7. The sensor assembly of claim 6, wherein the flexible NS connector tail is flexibly attached to the NIRS sensor body portion and is positioned proximate a midpoint between the pair of end surfaces.

8. The sensor assembly of claim 1, wherein the electrode sensor portion first segment having a body defined at least in part by a contact surface, an opposite rear surface, a plurality of lateral side surfaces, and a plurality of end surfaces;
wherein the flexible FES connector tail extends outwardly from the rear surface at a middle position of the body.

9. The sensor assembly of claim 1, wherein the electrode sensor portion first segment includes a plurality of said electrodes disposed along an axis, and the electrode sensor portion first segment is configured for attachment to a patient forehead.

10. The sensor assembly of claim 9, wherein the electrode sensor portion second segment includes a single said electrode, and the SES connector tail is configured to permit the electrode sensor portion second segment to be positioned for attachment to a patient temple area when the electrode sensor portion first segment is positioned for attachment to the patient forehead.

11. The sensor assembly of claim 1, wherein the electrode sensor portion is configured as an electroencephalography (“EEG”) sensor.

12. A physiological sensor assembly, comprising:
a near-infrared spectroscopy (NIRS) sensor portion having at least one light source, at least one light detector, a flexible NIRS sensor (NS) connector tail, and a first flex circuit, wherein the at least one light source and the at least one light detector are in signal communication with the first flex circuit, and the first flex circuit extends throughout the NS connector tail;
an electrode sensor portion having a plurality of electrodes configured to sense electrical activity of a patient, at least one flexible electrode sensor portion connector tail, and a second flex circuit, wherein the plurality of electrodes is in signal communication with the second flex circuit, and the second flex circuit extends throughout the at least one electrode sensor portion connector tail; and
a sensor assembly (SA) connector in communication with the first flex circuit and the second flex circuit;
wherein the flexible NS connector tail and the at least one flexible electrode sensor portion connector tail each extend outwardly from the SA connector independently of one another, and wherein the first flex circuit and the second flex circuit are in a stacked and folded configuration within the SA connector.

13. The sensor assembly of claim 12, wherein the flexible NS connector tail and the at least one flexible electrode sensor portion connector tail are independent of one another outside of the SA connector and the NIRS sensor portion and the electrode sensor portion are independently positionable relative to one another.

14. The sensor assembly of claim 13, wherein the electrode sensor portion includes a first segment having at least one said electrode, and a second segment having at least one said electrode, wherein the first segment and the second segment are independent of one another; and wherein the at least one flexible electrode sensor portion connector tail includes a flexible first electrode segment (FES) connector tail extending between the first segment and the SA connector, and a flexible second electrode segment (SES) connector tail extending between the second segment and the SA connector;
wherein the second flex circuit includes a first segment portion that extends through the FES connector tail and provides signal communication between the at least one said electrode of the first segment and the SA connector, and a second segment portion that extends through the SES connector tail and provides signal communication between the at least one said electrode of the second segment and the SA connector.

15. The sensor assembly of claim 12, wherein the first flex circuit and the second flex circuit are disposed in a stacked configuration and folded within the SA connector to form a folded portion.

16. The sensor assembly of claim 15, wherein the SA connector includes a stiffener panel having a bottom side surface, an opposing top side surface, and an insertion edge;
wherein the stiffener panel is disposed within the folded portion with the first flex circuit and the second flex circuit disposed contiguous with the bottom side surface and the top side surface and extending around the insertion edge.

17. A system for sensing a physiological parameter, comprising:
at least one physiological sensor assembly that includes:
a near-infrared spectroscopy (NIRS) sensor portion having at least one light source, at least one light detector, and a flexible NIRS sensor (NS) connector tail; and
an electrode sensor portion having a first segment, a second segment, and a plurality of electrodes configured to sense electrical activity of a patient, the first segment having at least one said electrode and a flexible first electrode segment (FES) connector tail, the second segment having at least one said electrode and a flexible second electrode segment (SES) connector tail; and
a sensor assembly (SA) connector in communication with the NIRS sensor portion, the electrode sensor portion first segment, and the electrode sensor portion second segment;
wherein the flexible NS connector tail, the flexible FES connector tail, and the flexible SES connector tail each extend outwardly from the SA connector independently of one another, and wherein the NIRS sensor portion, the electrode sensor portion first segment, and the electrode sensor portion second segment are independently positionable relative to one another; and
a base unit having a system controller in communication with the NIRS sensor portion, the electrode sensor portion, and a non-transitory memory storing instructions, which instructions when executed cause the system controller to:
control the NIRS sensor portion to control operation of the at least one light source and to receive signal communications from the at least one light detector;
control the electrode sensor portion to sense the electrical activity of the patient using the plurality of electrodes; and
process the signal communications from the at least one light detector and the sensed electrical activity of the patient to produce information relating to the physiological parameter.

18. The system of claim 17, wherein the NIRS sensor assembly includes a first flex circuit that extends through the NS connector tail, the first flex circuit having a plurality of first electrically conductive traces configured to provide electrical communication between the at least one light source and the SA connector, and configured to provide electrical communication between the at least one light detector and the SA connector.

19. The system of claim 18, wherein the electrode sensor portion includes a second flex circuit having a first segment portion that extends through the FES connector tail and a second segment portion that extends through the SES connector tail, the second flex circuit having a plurality of second electrically conductive traces configured to provide electrical communication between the electrode sensor portion first segment and the SA connector, and configured to provide electrical communication between the electrode sensor portion second segment and the SA connector.

20. The system of claim 19, wherein the first flex circuit and the second flex circuit are disposed in a stacked configuration and folded within the SA connector to form a folded portion.
